(19) United States
(12) Patent Application Publication
(10) Pub. No.: US 2018/0042961 A1
(43) Pub. Date: Feb. 15, 2018

(54) COMPOSITION AND METHOD FOR ALTERING HAIR PIGMENTATION

(71) Applicant: Kay Jay, LLC, Jackson, WY (US)

(72) Inventor: Kevin Meehan, Jackson, WY (US)

(21) Appl. No.: 15/792,498

(22) Filed: Oct. 24, 2017

Related U.S. Application Data
(62) Division of application No. 15/211,895, filed on Jul. 15, 2016, now abandoned.

(51) Int. Cl. A61K 33/34; A61K 9/00; A61K 8/362; A61K 31/405; A61K 31/225; A61K 8/37; A61K 31/194; A61K 8/19; A61K 8/49; A61K 8/44; A61K 8/41; A61Q 5/06; A61K 31/198

(52) U.S. Cl. CPC A61K 33/34; A61Q 5/065; A61K 9/0053; A61K 8/362; A61K 31/405; A61K 31/225; A61K 31/198; A61K 31/194; A61K 8/19; A61K 9/0014; A61K 8/492; A61K 8/447; A61K 8/41; A61K 8/37; A61K 2800/591; A61K 2800/92

(57) ABSTRACT

Achromotricia can begin due to a wide variety of reasons, one primarily being genetics, the IRF4 gene and exposures to various endogenous and exogenous factors. There are numerous stimuli currently recognized to influence the production of melanin by mature melanocytes. The composition presented herein recognizes the attributed principles which can influence the primary causes of achromotricia.

### CROSS REFERENCE TO RELATED APPLICATIONS

This application is a divisional of U.S. patent application Ser. No. 15/211,895, filed Jul. 15, 2016, which is incorporated herein in its entirety by reference.

### TECHNICAL FIELD

This disclosure generally relates to a composition and method for altering hair pigmentation. More particularly, this disclosure relates to a composition and method that activates melanin (eumelanin and pheomelanin) within the hair shaft follicle, increasing hair pigmentation, thus helping to eliminate gray hair.

### BACKGROUND

The demand in the marketplace for products which enhance a person's more youthful appearance are numerous and in high demand. Such products include wrinkle-fighting creams, teeth whiteners, Botox, laser treatments, and skin tightners. In addition to products, many procedures have been developed to assist men and women in their search for reversing the aging process, such as face lifts, liposuction, breast augmentation, chin augmentation, and arm lifts.

Hair coloring is an additional technique in the arsenal of methods to combat looking older. Both men and women experience hair turning gray, or even white. The reason hair turns gray is related to the lack of pigmentation in hair follicles. The pigmentation responsible for hair color is melanin. Melanin is made up of specialized pigment cells called melanocytes. As hair is being formed, melanocytes inject melanin into hair's keratin, which is the protein that makes up hair, skin, and nails.

Depending on a wide variety of factors, including a person's genetics and possibly environmental conditions, the loss of pigmentation in the hair follicle, also termed achromotricia, can occur at some point in a person's lifetime. For the majority of the population, achromotricia arises later in life. As a person ages, melanocytes lose their activity and slow down or cease inserting melanin into hair follicles. With the reduction of melanin, the hair turns gray and eventually white. The inventor has discovered the advantageous effect of utilizing combinations of the following components to slow or even reverse the graying process: Cysteine, Tryptophan, Tyrosine, Copper, Diacylglycerol, and/or Citric Acid.

### DETAILED DESCRIPTION OF EMBODIMENTS

The inventor has found that it would be beneficial to create a composition which activates melanin (eumelanin and pheomelanin) in the hair follicle shaft, thus diminishing gray hair. This disclosure relates to a composition which is postulated to slow damage or death to and/or reactive melanocytes, thus increasing melanin in the hair. The composition is comprised of Tyrosine, Cysteine, Copper, and Tryptophan, and optionally Citric Acid and/or Diacylglycerol.

Cysteine is the proteinogenic amino acid which counteracts the detrimental effects of acetaldehyde by conversion to acetic acid. Repeated and prolonged acetaldehyde exposure has demonstrated to be disadvantageous to the human body's health and the inventor recognizes the potential delirious effects to the melanocytes. As such, the addition of Cysteine to a composition will neutralize damage to the melanocytes thus increasing the presence of melanin in hair follicle shafts.

Tryptophan, 4-hydroxyphenylalanine, is also a proteinogenic amino acid. Tryptophan is a conditional amino acid. It functions as a primary building block for proteins, such as in the IRF4 protein. Tryptophan participates in the unique pentad repeat DNA-binding domain of Interferon Regulatory Factor 4 (“IRF4”). The IRF4 gene is strongly associated with pigmentation. Tryptophan is an indole derivative which can participate in the formation of leucodopachrome, an indolic intermediate in the melanogensis pathway. The following describes the pathway: Dopaquinone→Leucodopachrome→dopachrome→5,6-dihydroxyindole-2-carboxylic acid (optional)→quinone→eumelanin.

Tyrosine's oxidation pathway produces melanin. This pathway is paced by the rate-limiting enzyme, Tyrosinase, which contains the essential element copper. Tyrosine is a precursor to melanin via tyrosine's oxidation. The initial biosynthetic pathway for the melanin groups is catalyzed by the enzyme tyrosinase, noted by the following pathway: Tyrosine→Dopa→Dopaquinone.

Cysteine is a semi-conditional amino acid which plays a role in the formation of the pigment pheomelanin via the following pathways: Dopaquinone+Cysteine→5- or 2-cysteinyldopa→benzothiazine intermediate→pheomelanin.

Copper (Cu) participates in the catalytic reaction of the enzyme, Tyrosinase, the rate limiting enzyme which paces the production of melanin. Tyrosinase requires adequate amounts of copper to fulfill its role in the rate-limiting enzyme, Tyrosinase. Copper also participates as a component in the protein's superoxide dismutase, which decompose superoxide. Superoxide's potential of contributing to the destruction of animal cells, including melanocytes is remarkable.

Diacylglycerol is recognized as a second messenger signaling lipid. The diacylglycerol (diglyceride) is a precursor to arachindoyglycerol. There have been identified transport proteins for 2-arachindoyglycerol, which include the heat shock proteins (“HSP”). Intracellular HSP have been shown to have a protective function on the cell; in this proposed case, the melanocyte; allowing the cell to survive lethal conditions.

Citric acid is a tricarboxylic acid with antioxidant properties. Neutralizing hyperoxides (reactive oxygen species) is an important step in preserving the melanocyte cell function.

The inventor has recognized the combination of all of the above components or parts thereof result in unexpected and advantageous increased presence of melanin in hair follicles through activation of melanocyte activity.

According to some embodiments, the active components of the composition include ingredients such as Cysteine, Tryptophan, Tyrosine, and Copper. In embodiments, the active components of the composition include Cysteine, Tryptophan, Tyrosine, Copper, and optionally, Diacyglycerol.

Acceptable excipients for the composition include but are not limited to Citric Acid. The range of Citric Acid may vary from 0.05% of the total composition with no upper limit. Further, acceptable solvents include but are not limited to liquids or metal states, including salts.

Table 1A lists some of the ingredients for ingestible and/or topical application according to some of the exemplary embodiments of the invention as well as the concentration ranges for the components. As shown in Table 1A, the ingestible or topical composition may optionally include Cysteine, Trytophan, Tyrosine, Copper, Diacyglycerol, Citric Acid, or any combination thereof. The amount of the ingredients is given in concentration range or percentage by weight of the ingredient in the total composition.

TABLE 1A

| | Concentration |
|---|---|
| Component | Range (mcg) |
| Cysteine | from 0.05 mcg |
| Tryptophan | from 0.05 mcg |
| Tyrosine | from 0.05 mcg |
| Copper | from 0.05 mcg |
| Diacyglycerol | from 0.05 mcg |
| (diglyceride) (optional) | |
| Citric Acid (optional) | from 0.05 mcg |

As shown in Table 1A, the ingestible composition includes, at a minimum 0.05 mcg (or about 0.05 mcg) of Cysteine, 0.05 mcg (or about 0.05 mcg) of Tryptophan, 0.05 mcg (or about 0.05 mcg) of Tyrosine, and 0.05 mcg (or about 0.05 mcg) of Copper. There is no upper limit on the concentration ranges of the following components: Cysteine, Tryptophan, Tyrosine, and Copper.

In one embodiment, there is no upper limit to the amounts listed for any of the ingredients listed in Table 1A. As one skilled in the art would understand, the concentration range should be as high as a carrier will tolerate, which may be a one hundred percent composition and no solvent to the other extreme of broadening it to as low as a one-to-one ratio. Furthermore, as noted in the description and table, not all of the ingredients of Table 1A need to be used in the composition. For example, in one embodiment, the composition may include only Cysteine, Tryptophan, Tyrosine, Copper, and Diacylglycerol. In yet another embodiment, the active ingredients are comprised of Cysteine, Tryptophan, Tyrosine, Copper, and Citric Acid. In another embodiment, the composition comprises Cysteine, Tryptophan, Tyrosine, Copper, Diacylglycerol, and Citric Acid.

To obtain a solution for topical application, the ranges stated in table 1A can be converted into mcg/mL solutes which are blended into any acceptable solvents (distilled water, alcohols, glycerin, etc.).

The above descriptions are merely some examples of concentrations and capabilities available. No limitation to any particular embodiment is intended nor should be implied. Different processes may be separated and/or combined differently within the scope of embodiments.

The basic principles of producing or compounding this composition can be followed in a variety of methods; utilizing different mediums as solvents for the solution; powder, granular, or liquid. The final composition may be a capsule, compressed tablet, cream, or solution in a liquid solvent medium. The composition may be ingested or administered topically in any deliverable form in a mammal.

It will be appreciated that several of the above-disclosed and other features and functions, or alternatives thereof, may be desirably combined into many other different compositions, applications, and methods. Also that various presently unforeseen or unanticipated alternatives, modifications, variations, or improvements therein may be subsequently made by those skilled in the art which are also intended to be encompassed by the embodiments here.

While this invention has been particularly shown and described with reference to exemplary embodiments thereof, it will be understood by those skilled in the art that various changes in form and details may be made therein without departing from the spirit and scope of the embodiments described therein.

What is claimed is:

1. A method for altering hair pigmentation, the method comprising:
delivering to a subject an ingestible delivery form of a pharmaceutical composition comprising therapeutically effective amounts of cysteine, tryptophan, tyrosine, and copper; and
stimulating melanocyte activity within hair follicles of the subject, thereby altering the presence of melanin in the hair follicles of the subject.

2. The method of claim 1, wherein the pharmaceutical composition further comprises diacylglycerol.

3. The method of claim 1, wherein the pharmaceutical composition further comprises citric acid.

4. The method of claim 1, wherein a proportion by weight of the cysteine is at least about 0.05%.

5. The method of claim 1, wherein a proportion by weight of the tryptophan is at least about 0.05%.

6. The method of claim 1, wherein a proportion by weight of the tyrosine is at least about 0.05%.

7. The method of claim 1, wherein a proportion by weight of the copper is at least about 0.05%.

8. The method of claim 2, wherein a proportion by weight of the diacylglycerol is at least about 0.05%.

9. The method of claim 3, wherein a proportion by weight of the citric acid is at least about 0.05%.

10. A method for altering melanin in hair follicles, the method comprising:
delivering a plurality of active ingredients, comprising cysteine, tryptophan, tyrosine, and copper, in a vehicle for topical application to the skin of a mammal; and
stimulating melanocyte activity within hair follicles of the mammal, thereby altering hair pigmentation of the mammal.

11. The method of claim 10, wherein the pharmaceutical composition further comprises diacylglycerol.

12. The method of claim 10, wherein the pharmaceutical composition further comprises citric acid.

13. The method of claim 10, wherein a proportion by weight of the cysteine is at least about 0.05%.

14. The method of claim 10, wherein a proportion by weight of the tryptophan is at least about 0.05%.

15. The method of claim 10, wherein a proportion by weight of the tyrosine is at least about 0.05%.

16. The method of claim 10, wherein a proportion by weight of the copper is at least about 0.05%.

17. The method of claim 11, wherein a proportion by weight of the diacylglycerol is at least about 0.05%.

18. The method of claim 12, wherein a proportion by weight of the citric acid is at least about 0.05%.
